(19) United States
(12) Patent Application Publication
(10) Pub. No.: US 2026/0260749 A1
(43) Pub. Date: Sep. 3, 2026

(54) SYSTEMS AND METHODS FOR IDENTIFYING ATYPICAL HEMOLYTIC UREMIC SYNDROME (aHUS) PATIENTS USING CLINICAL FEATURES

(71) Applicant: Alexion Pharmaceuticals, Inc., Boston, MA (US)

(72) Inventors: Genevieve Helen Marie LYONS, Somerville, MA (US); Yan WANG, Lexington, MA (US)

(21) Appl. No.: 19/163,290

(22) PCT Filed: Mar. 7, 2024
(86) PCT No.: PCT/US2024/018807

§ 371 Date: Sep. 8, 2025

Related U.S. Application Data
(60) Provisional application No. 63/450,527, filed on Mar. 7, 2023.
(60) Provisional application No. 63/498,790, filed on Apr. 27, 2023.
(60) Provisional application No. 63/500,526, filed on May 5, 2023.

(51) Int. Cl. G16H 50/20; G16H 10/60

(52) U.S. Cl. CPC G16H 50/20; G16H 10/60

(57) ABSTRACT

Methods, systems, and computer readable media for identifying a patient cohort of patients that have atypical hemolytic uremic syndrome (aHUS) are provided. In aspects, diagnostic records and claims data are used to determine a subset of patients that may be used to train a machine learning model that may predict, based on a patient's claims data, the patient's aHUS diagnosis.

### CROSS-REFERENCE TO RELATED APPLICATION

This application claims the benefit of priority to U.S. Application No. 63/450.527, filed Mar. 7, 2023, U.S. Application No. 63/498,790, filed Apr. 27, 2023, and U.S. Application No. 63/500,526, filed May 5, 2023, which are incorporated by reference herein for all purposes.

### TECHNICAL FIELD

Embodiments of the present disclosure generally relate to computer implemented methods for identifying a patient cohort for patients having aHUS using machine learning methods.

### BACKGROUND

Atypical hemolytic uremic syndrome (aHUS) is a rare disease that is characterized by microangiopathic hemolytic anemia, thrombocytopenia, and renal damage. These characteristics can cause blood damage to the small blood vessels in an animal's organs, most often in the kidneys, which can lead to death if left untreated. Before 2022, there was no specific International Classification of Diseases (ICD) code for aHUS, and so there was no specific way of recording in a database that a patient had aHUS. This makes it difficult to appropriately identify patients to study aHUS outcomes.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 illustrates a method for identifying a patient cohort of patients having aHUS, according to some aspects.

FIG. 2 illustrates an example of a method for determining which patients may be included in a subset of patients according to inclusion criteria, according to some aspects.

FIG. 3 illustrates an example of a patient population used in training a machine learning model to predict ground-truth aHUS patients, according to some aspects.

FIG. 4 illustrates an example of a rule-based algorithm to identify aHUS patients, according to some aspects.

FIG. 5 illustrates an example of the performance of different machine learning algorithms in predicting ground-truth aHUS patients, according to some aspects.

FIG. 6 illustrates an example of the top relevant and non-relevant clinical features for predicting ground-truth aHUS patients, according to some aspects.

FIG. 7 illustrates an example of relevant and non-relevant clinical features for predicting ground-truth aHUS patients, according to some aspects.

FIG. 8 illustrates a block diagram of example components of a computer system, according to aspects of the present disclosure.

FIG. 9 illustrates a block diagram of a system for identifying a patient cohort of patients having aHUS, according to some aspects.

FIG. 10 illustrates an example of a system architecture for identifying a patient cohort of patients having aHUS, according to some aspects.

The present invention will be described with reference to the accompanying drawings. The drawing in which an element first appears is typically indicated by the leftmost digit(s) in the corresponding reference number.

### DETAILED DESCRIPTION

The lack of clinical classification codes related to aHUS has made it difficult to identify patients with aHUS (treated or untreated) in order to study aHUS outcomes over time. That is, existing databases may not contain information needed by previous identification systems to properly identify patients with aHUS. Further, many of the available patient databases do not include detailed diagnostic information from a medical professional, but rather simply include data from insurance claims made by a patient. Accordingly, aspects described herein endeavor to obtain a dataset of patients from such claims data that can be used for analyzing aHUS as a disease, thus retroactively identifying patients despite the patients' records not having aHUS-specific information therein.

Due to the lack of specific ICD diagnosis codes for aHUS, previous real-world studies using datasets were mainly focused on patients that were treated with specific treatments, such as eculizumab or ravulizumab. While this approach can help increase certainties in patient identification, it does not account for patients with aHUS that went untreated. To capture the aHUS landscape and assess existing unmet needs more comprehensively and accurately, it is important to include the untreated population. Because aHUS is a rare disease, even after an ICD diagnosis code is effective, it may take at least a year to capture enough aHUS patients to be used for analysis purposes. For these reasons, a machine learning (ML) algorithm to identify patients with aHUS may be developed and validated. In aspects of this disclosure, anonymous information from a database of electronic health records and insurance claims data may be used to develop a ML algorithm that can then be applied to patients in a claims database, in order to identify both treated and untreated patients with aHUS. These identified patients may then be added to a patient cohort. In some aspects, the patient cohort may be used in order to analyze aHUS outcomes. In some aspects, the patient cohort may be used in order to identify, treat, and/or follow up with patients having aHUS.

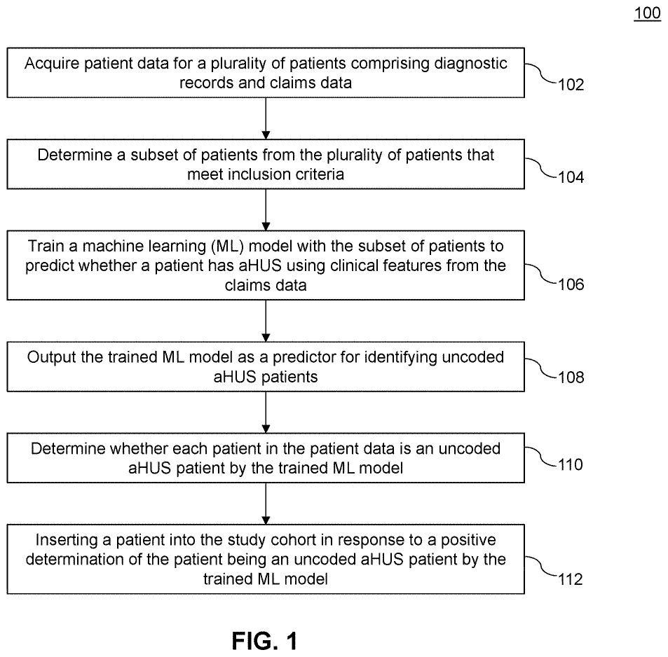

FIG. 1 illustrates a method 100 for identifying a patient cohort of patients having aHUS. The patient cohort may be used to study the landscape of aHUS and assess existing unmet needs more comprehensively and accurately with high quality real-world evidence and minimized potential bias. For example, the patient cohort may be used in claims data studies. The patient cohort may be a plurality of patients that had aHUS, whether they were treated, untreated, or partially treated. While the present disclosure uses aHUS as an example throughout, in some aspects method 100 may similarly be used to generate a patient cohort of patients for other rare diseases.

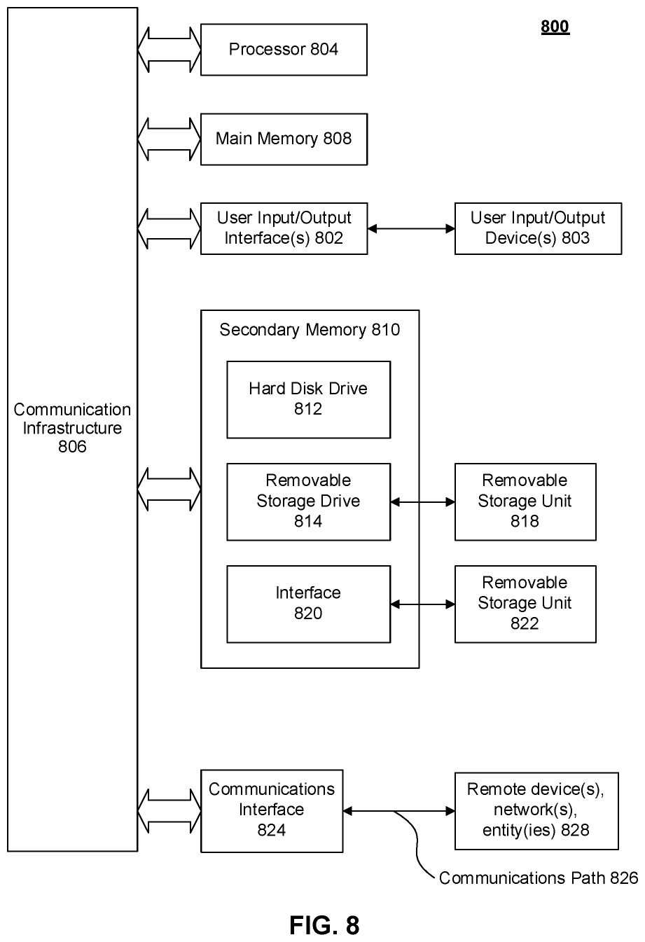

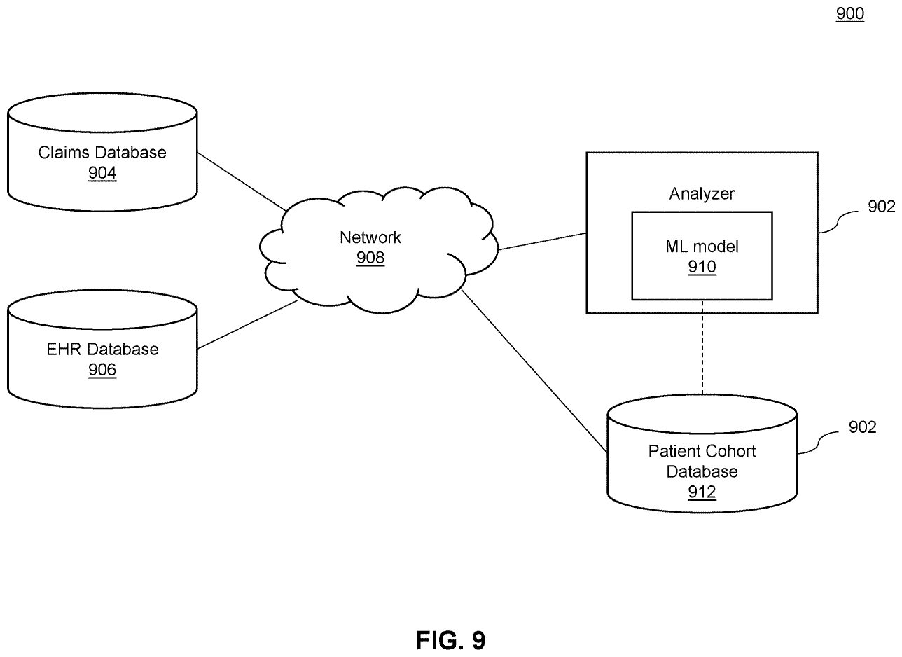

Method 100 will be described with reference to system 900 illustrated in FIG. 9, although other systems with similar functionality may alternatively be used. System 900 includes an analyzer 902. Analyzer 902 may be a processing device, such as computer system 800 described with reference to FIG. 8 below. Analyzer 902 is communicatively coupled to claims database 904 and electronic health record (EHR) database 906 via network 908. Claims database 904 may include claims data from, for example, insurance claims made by a plurality of patients. Data in claims database 904 may be anonymous data that has been deidentified and tokenized. Claims database 904 may be maintained by an insurance provider, and/or it may be a publicly available database. EHR database 906 may include electronic medical records from one or more sources, such as clinicians, hospitals, etc. Data in EHR database 906 may be anonymous data that has been deidentified and tokenized. The EHRs may include, among other notes, diagnostic information, other clinical information, clinician notes, and/or the like for a plurality of patients. In some aspects, claims database 904 and EHR database 906 may be co-located and/or maintained by the same entity; in other aspects, claims database 904 and EHR database 906 are separate from each other. In some aspects, each of claims database 904 and EHR database 906 may contain multiple distinct databases or data sets. In some aspects, claims database 904 and EHR database 906 are combined into the same database. In some aspects, one or both of claims database 904 and EHR database 906 are maintained in a distributed or cloud environment.

Network 908 may be of any suitable type, including individual connections via the internet such as cellular or WiFi networks. In some embodiments, network 908 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, or LAN. Because the information transmitted may be personal or confidential, security concerns may dictate one or more of these types of connections be encrypted or otherwise secured.

Network 908 may comprise any type of computer networking arrangement used to exchange data. For example, network 908 may be the internet, a private data network, virtual private network using a public network, and/or other suitable connection(s) that enables components in system 900 to send and receive information between the components of system 900. Network 908 may also include a public switched telephone network (“PSTN”) and/or a wireless network.

Analyzer 902 may include instructions stored thereon that, when executed, cause analyzer 902 to generate, train, and/or operate a ML algorithm to identify patients with aHUS. Such instructions, when executed, are referred to herein as ML model 910. ML model 910 may operate using data from one or more of claims database 904 or EHR database 906. Once trained, ML model 910 may reside within analyzer 902, or ML model 910 may be stored and/or executed separately from analyzer 902, such as on a third party system.

Patients who are identified by ML model 910 as having had or currently having aHUS may be added to a patient cohort. Information regarding patients in such a patient cohort may be stored in patient cohort database 912. Patient cohort database 912 may be a database maintaining a variety of data about one or more patients identified as having aHUS. For example, patient cohort database 912 may include claims data for one or more patients identified as having aHUS. In some aspects, patient cohort database 912 may include EHR data from one or more patient identified as having aHUS for which EHR data exists in EHR database 906. Patient cohort database 912 may contain anonymous data that has been deidentified and tokenized so as to ensure privacy and security. Patient cohort database 912 may be accessible to ML model 910 via network 908; alternatively, patient cohort database 912 may be directly connected to analyzer 902 or other device containing ML model 910. In some aspects, patient cohort database 902 is co-located with analyzer 902, such as on the same computing device as analyzer 902.

Returning to FIG. 1, method 100 may be performed by, for example, analyzer 902. At step 102, patient data for a plurality of patients is acquired. Patient data may be acquired from, for example, claims database 904 and EHR database 906. The patient data may include claims data. In some aspects, the patient data may include diagnostic records and claims data. In some aspects, the patient data may include data from patients that have diseases related to chronic kidney disease. Specifically, the diagnostic data may include data from patients with aHUS-related diagnoses, including, but not limited to, patients diagnosed with hemolytic uremic syndrome (HUS), HUS-related hemolytic anemia, or thrombotic microangiopathy (TMA). Similarly, the claims data may include patients with aHUS related diagnosis codes, including, but not limited to, HUS diagnosis codes, HUS-related hemolytic anemia codes, or TMA codes. The patient data may be acquired from any claims database that includes both diagnostic records and claims data. For example, patient data may be acquired from Optum's de-identified Market Clarity Data (OMCD). In such example, the patient data has data for 5,800,383 patients. In some aspects, the patient data may include data from a specific date range (e.g. from Jan-2016 to Jan-2022).

The diagnostic records may be records collected or created by a physician during a patient's diagnosis and treatment. In some aspects, the diagnostic records may be de-identified and tokenized electronic health records. The claims data may be insurance claims data, such as medical and/or prescription claims data, which provides each patient's medical history. The claims data may include clinical features of each patient. The clinical features may include, for example, comorbidities, medications, procedures, and tests for each patient. Comorbidities include, for example, all existing diagnoses, complications post-HUS/TMA diagnosis or treatment, and/or renal disease progression following a HUS/TMA diagnosis. Medications may include medications taken prior to a HUS/TMA diagnosis and up to a specific time period, such as a year, following a HUS/TMA diagnosis. Procedures and tests may include any procedure or test completed for a patient during HUS/TMA treatment and following treatment. For example, a patient's clinical features may indicate that a patient underwent ADAMTS13 testing and dialysis.

At step 104, a subset of patients is determined from the patient data based on inclusion criteria. The inclusion criteria determines which patients may be used to train a machine learning model. In some aspects of this disclosure, the inclusion criteria includes a patient having both claims data including an aHUS related code and diagnostic records including an aHUS related diagnosis. Because ICD codes did not exist prior to 2022 for aHUS diagnosis, the only indication of aHUS diagnosis in claims data may be a patient's full treatment of aHUS, which excludes patients that went untreated. Therefore, the inclusion criteria may also include diagnostic records to establish a definitive, ground-truth diagnosis of each patient.

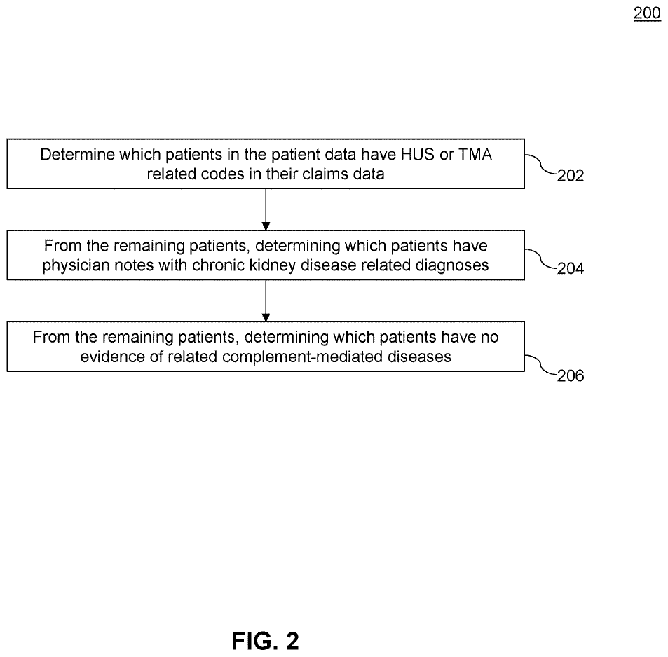

FIG. 2 describes an example of a method 200 that may be used to determine which patients may be included in the subset of patients according to the inclusion criteria, according to some aspects of this disclosure. A skilled artisan will recognize that other inclusion criteria may be used to optimize the subset of patients for a particular use case. At step 202, it is determined which patients in the plurality of patients have claims data that includes a certain number of related diagnosis codes within a given time period. For example, it may be determined which patients in the plurality of patients have claims data that includes ≥2 hemolytic uremic syndrome diagnosis codes (e.g., ICD-10, D59.3) and HUS-related hemolytic anemia codes (e.g., ICD-10, D59.4, D59.9) or ≥2 thrombotic microangiopathy (TMA) diagnosis codes (e.g., ICD-10, M31.1) within 6 months of the patient's index date. The index date is the date of the first HUS or TMA related diagnosis code in a patient's record. In an experimental test, when using the OMCD, step 202 reduced the number of patients from 5,800,383 patients to 7,827 patients.

At step 204, it is determined whether each remaining patient has information in their diagnostic record corresponding to a related disease diagnosis within a given time period. For example, it may be determined which of the remaining patients has data from ≥2 physician notes between 2 weeks before and 1 year after the patient's index date suggesting a chronic kidney disease diagnosis or related issues. The physician notes may be from the diagnostic records in the patient data. In the experimental test, when using OMCD, step 204 reduced the number of patients from 7,827 to 2,243 patients.

At step 206, patients with evidence of complement-mediated diseases in a given time period are removed from the data set. For example, it may be determined whether each remaining patient has no evidence of other complement-mediated diseases within 6 months before or after the patient's index date. In the experimental test, when using OMCD, step 206 reduced the number of patients from 2,243 to 1,992 patients. These remaining patients may constitute the subset of patients that will be used to train a machine learning model, according to further aspects.

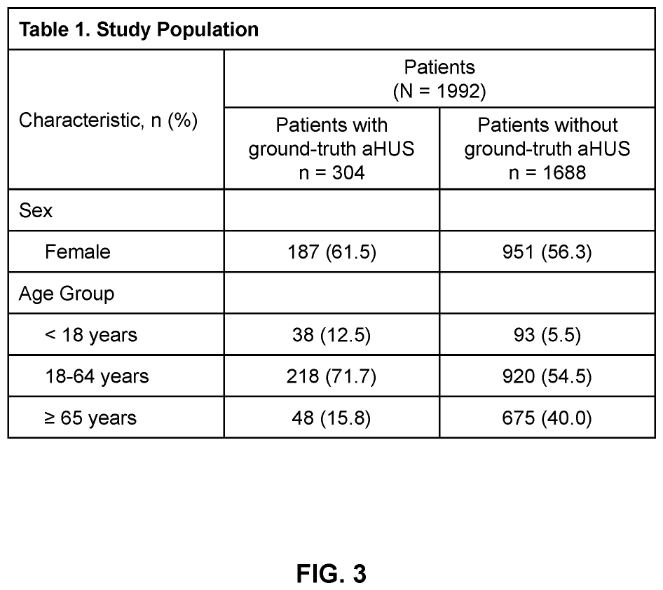

For the purposes of training a machine learning model, both ground-truth aHUS patients and ground-truth non-aHUS patients may be included in the subset of patients. Ground-truth aHUS diagnosis may be established using, for example, tokenized physician notes from the diagnostic records. This may be performed using, for example, natural language processing (NLP). In the experimental test discussed above, ground-truth aHUS was established for patients with the term ‘aHUS’ or ‘atypical hemolytic uremic syndrome’ mentioned in ≥2 physician notes, without negative sentiment (such as ‘aHUS unlikely’) and without positive mention of thrombotic thrombocytopenia purpura (TTP). In the experimental test, ground-truth aHUS also included confirmed full treatment (≥5 eculizumab or ≥2 ravulizumab doses) of patients with aHUS. In the experimental test, a ground-truth non-aHUS patient was determined when there was no evidence of a positive aHUS diagnosis without TTP in ≥2 physician notes and no confirmed treatment within 2 years of the patient's index date. In the experimental test using OCMD as the patient data, of the 5,800,383 patient records screened, 1,992 patient records met the inclusion criteria for the subset of patients. Of the 1,992 patients included in the subset of patients, 304 (15%) had ground-truth aHUS based on physician notes (see FIG. 3).

Returning to FIG. 1, at step 106, a machine learning (ML) model is trained using the subset of patients to predict whether a patient in the subset of patients has aHUS using clinical features from the claims data as the features in the ML model and the target is the aHUS related diagnosis from the diagnostic records. A ML model is a computer program that uses statistical techniques to identify patterns in data and predict outputs. The ML model may be, for example, ML model 910 of analyzer 902 as illustrated in FIG. 9.

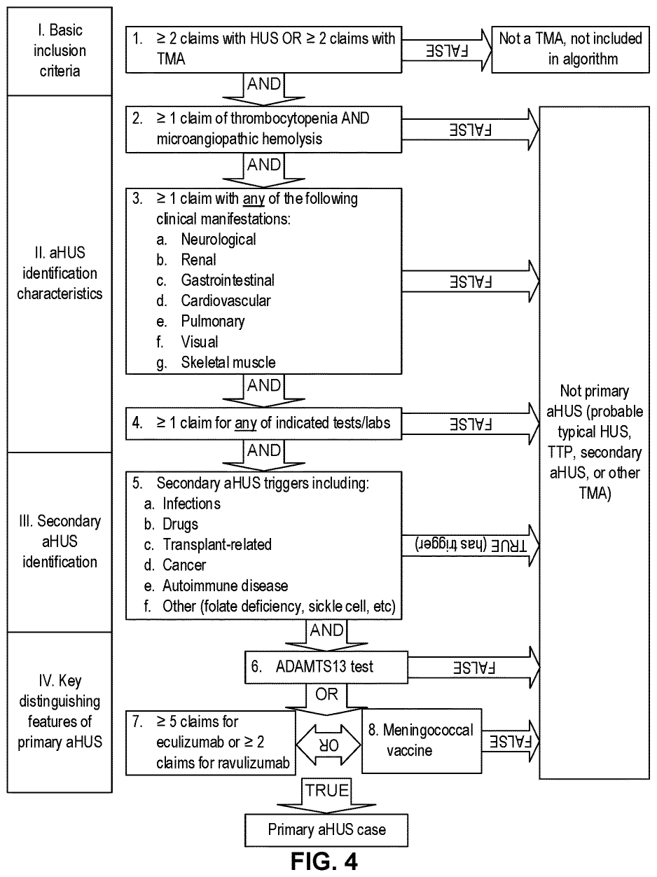

Previous attempts to develop a patient cohort database used a rule-based algorithm instead of a ML model. The rule-based algorithm was based on a theoretical patient journey that incorporated several claims-based criteria for the identification of aHUS, secondary aHUS, and key distinguishing features of aHUS (see FIG. 4). However, due to a lack of laboratory results to enable differential diagnosis, the rule-based algorithm was unable to provide clear differences between TMA types, specifically between patients with and without aHUS. Further, the rule-based algorithm was unable to identify untreated patients.

Accordingly, according to aspects of the present disclosure, a ML model may be used to predict patients with aHUS. In some aspects, one or more ML algorithms (e.g. elastic net, classification and/or regression trees, and/or random forest, alone or in combination) may be developed for the ML model using clinical features (e.g., renal, pulmonary, cardiovascular, hematology and/or others) extracted from the claims data before and after the index date. In some aspects, a portion of the subset of patients is used to train the one or more ML algorithms and a portion is reserved for testing (e.g. 70% for training and 30% for testing). In some aspects, the ML model may be an ensemble ML algorithm, in which confirmed treatment history, such as ground-truth aHUS diagnosis, from the diagnostic records is combined with the one or more ML algorithms to train and test the one or more ML algorithms. In some aspects, all clinical features available are used by the ML model to predict aHUS outcomes. In other aspects, clinical features that are identified as the most important, or predictive, may be used by the ML model to predict aHUS outcomes. For example, χ-square test and p<0.05 may be used by the ML model to predict aHUS outcomes.

In some aspects, one or more ML models may be developed with two or more different probability thresholds to allow for the selection of optimal sensitivity, specificity, and positive predictive value (PPV) metrics. Sensitivity relates to the proportion of patients with aHUS who were correctly predicted by the ML model. Specificity relates to the proportion of patients without aHUS who were correctly predicted by the ML model. PPV relates to the proportion of patients who were predicted to have aHUS who did have confirmed aHUS. In some aspects, the ML model with the highest PPV may be selected to maximize the precision of the prediction.

Accuracy metrics of the ML model may be calculated using ground-truth aHUS compared to predicted aHUS. In some aspects, the accuracy metrics include sensitivity, specificity, PPV, and area under the curve (AUC) related to the identification of aHUS. The sensitivity is related to the identification of treated or untreated aHUS patients, which is calculated using patients with or without a record of partial treatment (e.g., ≥1 eculizumab or ≥1 ravulizumab doses) after the index date, respectively.

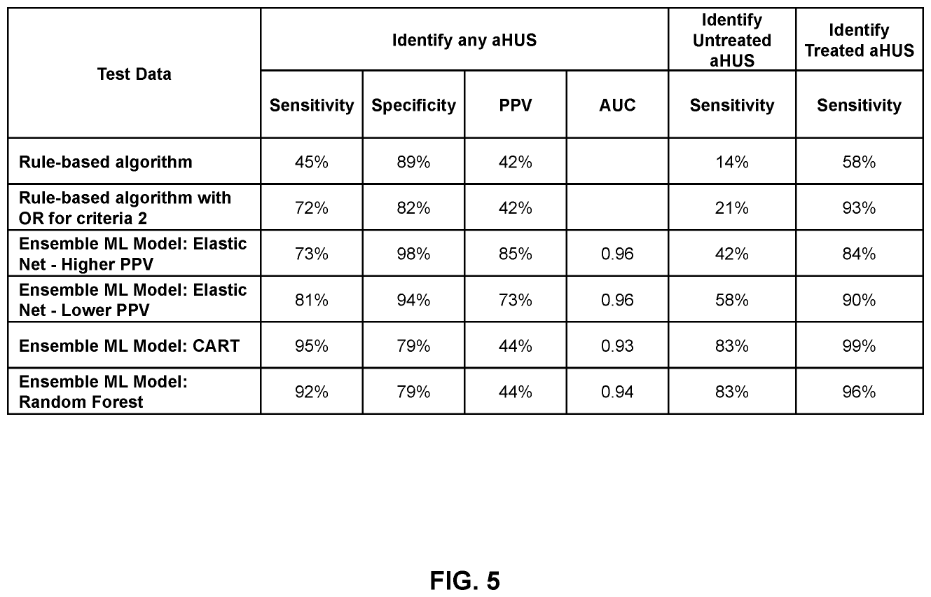

FIG. 5 illustrates results of an experimental test executed based on some aspects of the present disclosure. In the experimental test, the elastic net ML algorithm had the highest positive predictive value (85%) for identifying aHUS, with 73% sensitivity and 98% specificity. Elastic net is a variation of logistic regression, but elastic net uses regularization to handle the many thousands of features that are available in the claims data. For example, the OMCD claims data includes 1,286 total features. The elastic net algorithm is especially more powerful when a training set has many features and a low sample size, which is the case with a dataset of patients with a rare disease (e.g. aHUS). The elastic net algorithm is therefore the selected algorithm for further discussion so as to maximize the precision of the prediction.

In addition, several clinical features (renal, pulmonary, cardiovascular, hematological, and others) were associated with either an increased or decreased chance of being identified as aHUS. These features are associations, but are not causal. The most important features that the ML algorithm in the experimental test used to identify patients with aHUS were hospitalization, renal disease progression, cardiovascular/pulmonary complications, and the absence of bacterial infections.

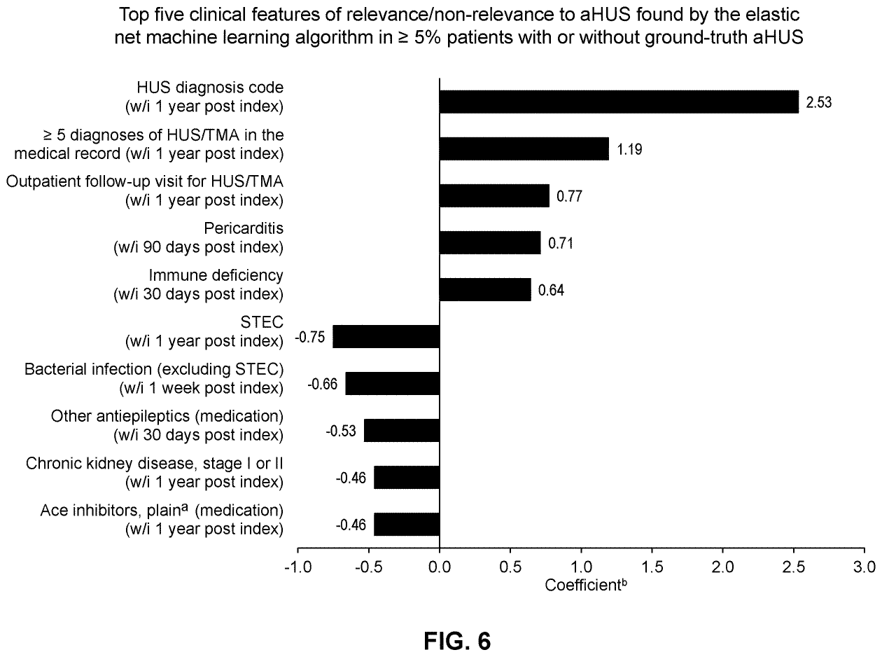

FIG. 6 illustrates specific features that were found to have the most relevance to the ML algorithm's predictions and were clinically relevant, according to aspects of the present disclosure. Such features include, but are not limited to, HUS diagnosis codes, ≥5 diagnoses of HUS/TMA in the medical record of the patient, outpatient follow-up visits for HUS/TMA, pericarditis, and immune deficiency. Clinical features that were found to have non-relevance include, but are not limited to, Shiga toxin-producing E. coli (STEC) infections, bacterial infections (excluding STEC), other antiepileptics (medication), chronic kidney infections (CKD) stage I or II, and plain ACE inhibitors (medication). These clinical features, as seen in FIG. 6, have coefficients whose magnitude indicates the relative weight of the respective feature for prediction in the ML algorithm. A positive coefficient indicates that the feature has relevance because that feature is more frequently seen in patients with aHUS. A negative coefficient indicates that the feature has non-relevance because that feature is more frequently seen in patients without aHUS or who have been diagnosed with TMA or HUS but do not have ground-truth aHUS. Both relevant and non-relevant features may be used to train and/or apply the ML model. According to aspects herein, the ML model may use one, multiple, or all relevant features in its analysis. According to aspects herein, the ML model may use one, multiple, or all non-relevant features in its analysis. According to aspects herein, the ML model may use one, multiple, or all relevant and non-relevant features in its analysis.

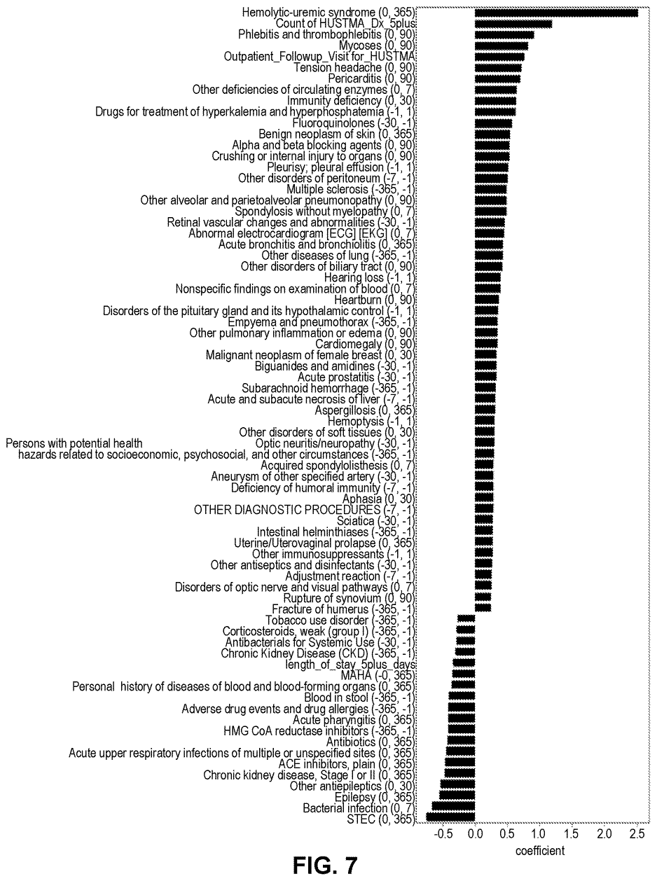

Additional features are identified in FIG. 7. In some aspects, features having a relevant association to a positive aHUS diagnosis include: multiple concordant HUS diagnoses, end-stage renal disease (ESRD), dialysis, pleurisy, lung disease, alveolar pneumopathy, pericarditis, beta blockers, cardiomegaly, thrombophlebitis, hyperphosphatemia, immune deficiency, and multiple sclerosis. In some aspects, features having a relevant association to a negative aHUS diagnosis include: CKD Stage I/II, non-autoimmune hemolytic anemia, history of blood diseases, epilepsy, STEC, bacterial infections, antibiotic use, and obesity. It is to be appreciated that any one or combination of two, more, or all of the clinically relevant clinical features in FIG. 7 (positive, negative, or both) may be used as features to predict an aHUS diagnosis of a patient.

Returning to FIG. 1, at step 108, the trained ML model is output as a predictor for identifying uncoded aHUS patients for inclusion in a patient cohort. An uncoded aHUS patient may be any patient in the patient data that had aHUS (treated or untreated) but does not have an aHUS ICD code in their claims data.

At step 110, the trained ML model may be used to determine whether each patient in the patient data is an uncoded aHUS patient.

At step 112, in response to a positive determination of a patient being an uncoded aHUS patient by the trained ML model, the patient is inserted into the patient cohort. The patient cohort may also include the subset of patients identified as ground-truth aHUS patients in step 104.

This ML approach addresses the limitations of rule-based algorithms, potentially leading to the identification of more patients with aHUS with greater accuracy from claims data and an improved understanding of the aHUS treatment landscape. The ML approach also addresses the rule-based algorithms' inability to identify untreated patients. In aspects, when the ML model uses an elastic net ML algorithm, the elastic net ML algorithm permits more accurate identification of a larger group of patients with aHUS in large databases, such as those with undiagnosed or untreated aHUS, especially in claims-based data analysis, which could provide an improved understanding of the treatment of patients with aHUS.

Additionally, the ML model may be used to identify a patient, or subject, that is suffering from or is likely to suffer from aHUS using only their claims data. This may be done by an analyzer, such as analyzer 902, or other computer system (e.g. computer system 800) receiving data that includes the subject's clinical features (e.g. renal, pulmonary, cardiovascular, or hematology features, or a combination thereof). The ML model may then generate a PPV score from the subject's clinical features. If the PPV score is above a threshold value (e.g. p<0.05), the subject may be identified as suffering or likely to suffer from aHUS.

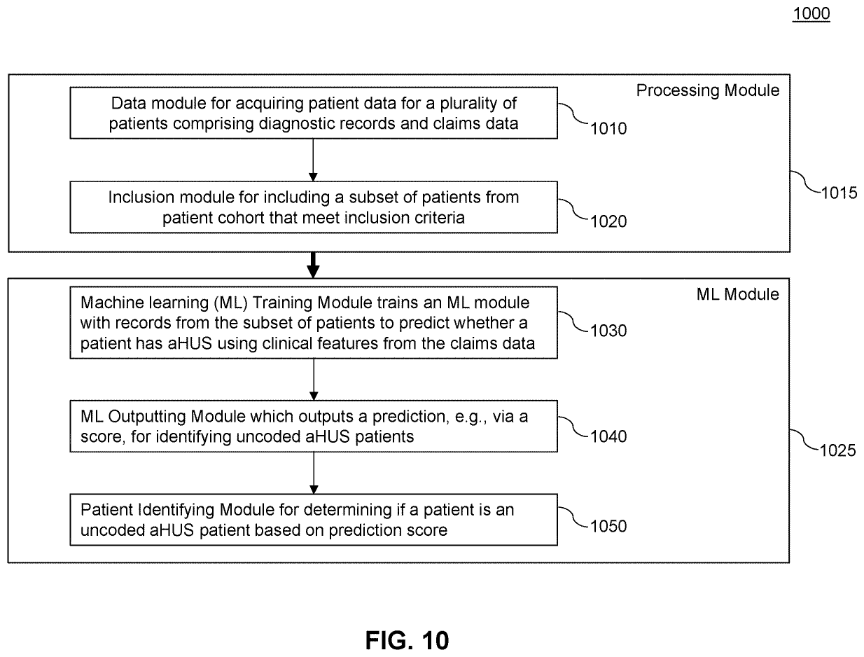

FIG. 10 illustrates an example of a system architecture 1000 for identifying a patient cohort of patients having aHUS, according to some aspects. A memory, for example, main memory 808, may be adapted to include one or more software modules (a set of instructions that is executed by a processor). In particular, the memory may include processing module 1015 and ML module 1025. Processing module 1015 may include data module 1010 and inclusion module 1020. ML module 1025 may include ML training module 1030, ML outputting module 1040, and patient identifying module 1050.

Data module 1010 may acquire patient data for a plurality of patients comprising diagnostic records and claims data. For example, data module 1010 may execute functionality as described with respect to step 102. Inclusion module 1020 may then determine a subset of patients from the patient cohort that meet inclusion criteria. For example, inclusion module 1020 may execute functionality as described with respect to step 104.

ML training module 1030 may train an ML model with diagnostic records from the subset of patients to predict whether a patient has aHUS using clinical features from the claims data. For example, ML training module 1030 may execute functionality as described with respect to step 106. ML outputting module 1040 may output a prediction via a prediction score for identifying uncoded aHUS patients. For example, ML outputting module 1040 may execute functionality as described in step 108. Patient identifying module 1050 may determine whether a patient is an uncoded aHUS patient based on the prediction score of the patient. For example, patient identifying module 1050 may execute functionality as described in steps 110 and 112.

FIG. 8 shows a computer system 800, according to some aspects. Various aspects and components therein, such as analyzer 902 and/or ML model 910, method 100, and/or method 200, can be implemented, for example, using computer system 800 or any other well-known computer systems, such that the computer system, when programmed according to aspects described herein, becomes a special purpose machine.

In some aspects, computer system 800 can comprise one or more processors (also called central processing units, or CPUs), such as a processor 804. Processor 804 can be connected to a communication infrastructure or bus 806.

In some aspects, one or more processors 804 can each be a graphics processing unit (GPU). In some aspects, a GPU is a processor that is a specialized electronic circuit designed to process mathematically intensive applications. The GPU can have a parallel structure that is efficient for parallel processing of large blocks of data, such as mathematically intensive data common to computer graphics applications, images, videos, etc.

In some aspects, computer system 800 can further comprise user input/output device(s) 803, such as monitors, keyboards, pointing devices, etc., that communicate with communication infrastructure 806 through user input/output interface(s) 802. Computer system 800 can further comprise a main or primary memory 808, such as random access memory (RAM). Main memory 808 can comprise one or more levels of cache. Main memory 808 has stored therein control logic (i.e., computer software) and/or data.

In some aspects, computer system 800 can further comprise one or more secondary storage devices or memory 810. Secondary memory 810 can comprise, for example, a hard disk drive 812 and/or a removable storage device or drive 814. Removable storage drive 814 can be a floppy disk drive, a magnetic tape drive, a compact disk drive, an optical storage device, tape backup device, and/or any other storage device/drive. Removable storage drive 814 can interact with a removable storage unit 818. Removable storage unit 818 can comprise a computer usable or readable storage device having stored thereon computer software (control logic) and/or data. Removable storage unit 818 can be a floppy disk, magnetic tape, compact disk, DVD, optical storage disk, and/any other computer data storage device. Removable storage drive 814 reads from and/or writes to removable storage unit 818 in a well-known manner.

In some aspects, secondary memory 810 can comprise other means, instrumentalities or other approaches for allowing computer programs and/or other instructions and/or data to be accessed by computer system 800. Such means, instrumentalities or other approaches can comprise, for example, a removable storage unit 822 and an interface 820. Examples of the removable storage unit 822 and the interface 820 can comprise a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

In some aspects, computer system 800 can further comprise a communication or network interface 824. Communication interface 824 enables computer system 800 to communicate and interact with any combination of remote devices, remote networks, remote entities, etc. (individually and collectively referenced by reference number 828). For example, communication interface 824 can allow computer system 800 to communicate with remote devices 828 over communications path 826, which can be wired and/or wireless, and which can comprise any combination of LANs, WANs, the Internet, etc. Control logic and/or data can be transmitted to and from computer system 800 via communications path 826.

In some aspects, a non-transitory, tangible apparatus or article of manufacture comprising a non-transitory, tangible computer useable or readable medium having control logic (software) stored thereon is also referred to herein as a computer program product or program storage device. This includes, but is not limited to, computer system 800, main memory 808, secondary memory 810, and removable storage units 818 and 822, as well as tangible articles of manufacture embodying any combination of the foregoing. Such control logic, when executed by one or more data processing devices (such as computer system 800), causes such data processing devices to operate as described herein.

Based on the teachings contained in this disclosure, it will be apparent to those skilled in the relevant art(s) how to make and use aspects of this disclosure using data processing devices, computer systems and/or computer architectures other than that shown in FIG. 8. In particular, aspects described herein can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

Aspects of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The disclosure relates to the following non-limiting aspects:

Aspect 1: A method for identifying a patient cohort of patients that have atypical hemolytic uremic syndrome (aHUS), comprising:
acquiring, by one or more processors, patient data for a plurality of patients, wherein the patient data comprises diagnostic records comprising an aHUS related diagnosis of each patient in the plurality of patients and claims data comprising clinical features of each patient in the plurality of patients;
determining, by the one or more processors, a subset of patients from the plurality of patients that meet inclusion criteria, wherein the inclusion criteria comprises:
claims data comprising an aHUS related code, and
diagnostic records comprising an aHUS related diagnosis;
training, by the one or more processors, a machine learning model using the subset of patients to predict whether a patient in the subset of patients has aHUS, wherein features of the machine learning model correspond to the clinical features in the claims data; and
outputting, by the one or more processors, the trained machine learning model as a predictor for identifying uncoded aHUS patients for inclusion in the patient cohort.

Aspect 2: The method of any one of the foregoing or following Aspects, further comprising:
determining, by the trained machine learning model, whether each patient in the patient data is an uncoded aHUS patient; and
in response to a positive determination of a patient being an uncoded aHUS patient by the trained ML model, inserting, by the one or more processors, the patient into the patient cohort.

Aspect 3: The method of any one of the foregoing or following Aspects, wherein the diagnostic records are tokenized electronic health records.

Aspect 4: The method of any one of the foregoing or following Aspects, wherein the clinical features comprise renal features, pulmonary features, cardiovascular features, hematology features, or a combination thereof.

Aspect 5: The method of any one of the foregoing or following Aspects, wherein the aHUS related diagnosis is at least one of hemolytic uremic syndrome (HUS) diagnosis, HUS-related hemolytic anemia diagnosis, or thrombotic microangiopathy (TMA) diagnosis.

Aspect 6: The method of any one of the foregoing or following Aspects, wherein the aHUS related code is at least one of hemolytic uremic syndrome (HUS) codes, HUS-related hemolytic anemia codes, or thrombotic microangiopathy (TMA) codes.

Aspect 7: The method of any one of the foregoing or following Aspects, wherein the machine learning model comprises an elastic net algorithm.

Aspect 8: The method of any one of the foregoing or following Aspects, particularly Aspect 7, wherein the features of the machine learning model comprise at least one of hospitalizations, progression of renal disease, cardiovascular complications, pulmonary complications, and absence of bacterial infections.

Aspect 9: The method of any one of the foregoing or following Aspects, particularly Aspect 8, wherein the features of the machine learning model comprise hospitalizations, progression of renal disease, cardiovascular complications, pulmonary complications, and absence of bacterial infections.

Aspect 10: The method of any one of the foregoing or following Aspects, particularly Aspect 9, wherein the features of the machine learning model consist of hospitalizations, progression of renal disease, cardiovascular complications, pulmonary complications, and absence of bacterial infections.

Aspect 11: The method of any one of the foregoing or following Aspects, wherein the features of the machine learning model comprise at least one of a HUS diagnosis code, a threshold number of diagnoses of HUS/TMA in the diagnostics records, occurrence of an outpatient follow-up visit for HUS/TMA, pericarditis, or immune deficiency.

Aspect 12: The method of any one of the foregoing or following Aspects, particularly Aspect 11, wherein the features of the machine learning model further comprise at least one of STEC, a bacterial infection, antiepileptics medication, chronic kidney disease, or ACE inhibitors.

Aspect 13: The method of any one of the foregoing or following Aspects, particularly Aspect 11, wherein the features of the machine learning model comprise a HUS diagnosis code, a threshold number of diagnoses of HUS/TMA in the diagnostics records, occurrence of an outpatient follow-up visit for HUS/TMA, pericarditis, and immune deficiency.

Aspect 14: The method of any one of the foregoing or following Aspects, particularly Aspect 13, wherein the features of the machine learning model further comprise STEC, a bacterial infection, antiepileptics medication, chronic kidney disease, and ACE inhibitors.

Aspect 15: The method of any one of the foregoing or following Aspects, particularly Aspect 13, wherein the features of the machine learning model consist of a HUS diagnosis code, a threshold number of diagnoses of HUS/TMA in the diagnostics records, occurrence of an outpatient follow-up visit for HUS/TMA, pericarditis, and immune deficiency.

Aspect 16: The method of any one of the foregoing or following Aspects, particularly Aspect 15, wherein the features of the machine learning model consist of a HUS diagnosis code, a threshold number of diagnoses of HUS/TMA in the diagnostics records, occurrence of an outpatient follow-up visit for HUS/TMA, pericarditis, immune deficiency, STEC, a bacterial infection, antiepileptics medication, chronic kidney disease, and ACE inhibitors.

Aspect 17: The method of any one of the foregoing or following Aspects, wherein the features of the machine learning model comprise at least one of STEC, a bacterial infection, antiepileptics medication, chronic kidney disease, or ACE inhibitors.

Aspect 18: The method of any one of the foregoing or following Aspects, particularly Aspect 17, wherein the features of the machine learning model comprise STEC, a bacterial infection, antiepileptics medication, chronic kidney disease, and ACE inhibitors.

Aspect 19: The method of any one of the foregoing or following Aspects, particularly Aspect 18, wherein the features of the machine learning model consist of STEC, a bacterial infection, antiepileptics medication, chronic kidney disease, or ACE inhibitors.

Aspect 20: The method of any one of the foregoing or following Aspects, wherein the positive determination is determined by a positive predictive value that is above a threshold value.

Aspect 21: A system for identifying a patient cohort of patients that have atypical hemolytic uremic syndrome (aHUS), comprising:
at least one processor; and
a memory having instructions stored thereon that, when executed by the at least one processor, cause the at least one processor to:
acquire patient data for a plurality of patients, wherein the patient data comprises diagnostic records comprising an aHUS related diagnosis of each patient in the plurality of patients and claims data comprising clinical features of each patient in the plurality of patients;
determine a subset of patients from the plurality of patients that meet inclusion criteria, wherein the inclusion criteria comprises:
claims data comprising an aHUS related code, and
diagnostic records comprising an aHUS related diagnosis;
train a machine learning model using the subset of patients to predict whether a patient in the subset of patients has aHUS, wherein the features of the machine learning model correspond to the clinical features in the claims data and the target is the aHUS related diagnosis; and
output the machine learning model as a predictor for identifying uncoded aHUS patients in claims data for inclusion in the patient cohort.

Aspect 22: The system of any one of the foregoing or following Aspects, particularly Aspect 20, wherein the instructions further cause the at least one processor to: determine whether each patient in the plurality of patients is an uncoded aHUS patient; and in response to a positive determination of a patient being an uncoded aHUS patient, insert the patient into the patient cohort.

Aspect 23: The system of any one of the foregoing or following Aspects, particularly Aspect 21, wherein the diagnostic records are electronic health records.

Aspect 24: The system of any one of the foregoing or following Aspects, particularly Aspect 21, wherein the clinical features comprise renal features, pulmonary features, cardiovascular features, hematology features, or a combination thereof.

Aspect 25: The system of any one of the foregoing or following Aspects, particularly Aspect 21, wherein the aHUS related diagnosis is at least one of hemolytic uremic syndrome (HUS) diagnosis, HUS-related hemolytic anemia diagnosis, and thrombotic microangiopathy (TMA) diagnosis.

Aspect 26: The system of any one of the foregoing or following Aspects, particularly Aspect 21, wherein the aHUS related code is at least one of hemolytic uremic syndrome (HUS) codes, HUS-related hemolytic anemia codes, and thrombotic microangiopathy (TMA) codes.

Aspect 27: The system of any one of the foregoing or following Aspects, particularly Aspect 21, wherein the machine learning model comprises an elastic net algorithm.

Aspect 28: The system of any one of the foregoing or following Aspects, particularly Aspect 27, wherein the features of the machine learning model comprise at least one of hospitalizations, progression of renal disease, cardiovascular complications, pulmonary complications, and absence of bacterial infections.

Aspect 29: The system of any one of the foregoing or following Aspects, particularly Aspect 21, wherein the positive determination is determined by a positive predictive value that is above a threshold value.

Aspect 30: A method for identifying a patient cohort of patients that have atypical hemolytic uremic syndrome (aHUS), comprising:
predicting, by a machine learning model, whether each patient in a plurality of patients is an uncoded aHUS patient, wherein the machine learning model is trained to predict whether a patient has aHUS based on clinical features of the patient's claims data; and
in response to a positive prediction of a patient being an uncoded aHUS patient, inserting, by the one or more processors, the patient into the patient cohort.

Aspect 31: A system for identifying a subject that is suffering from or is likely to suffer from atypical hemolytic uremic syndrome (aHUS), the system comprising:
a non-transitory memory; and
a hardware processor coupled with the non-transitory memory and configured to read instructions from the non-transitory memory to cause the system to perform operations comprising:
receiving data comprising a subject's clinical features selected from renal features, pulmonary features, cardiovascular features, hematology features or a combination thereof; and
generating, using machine learning (ML) algorithm selected from elastic net, classification tree, and random forest, a positive predictive value (PPV) score from the subject's clinical features data, wherein if the PPV score is above a threshold value, the subject is identified as suffering from or being likely to suffer from aHUS.

Aspect 32: The system of any one of the foregoing or following Aspects, particularly Aspect 31, wherein the ML algorithm comprises elastic net and the clinical features data includes (1) hospitalization, (2) progression of renal disease, (3) cardiovascular/pulmonary complications, and/or (4) absence of bacterial infections.

Aspect 33: A non-transitory computer-readable medium (CRM) having stored thereon computer-readable instructions executable to cause a computer system to perform operations comprising: (i) receiving data comprising a subject's clinical features selected from renal features, pulmonary features, cardiovascular features, hematology features or a combination thereof, wherein the subject is suffering from or is likely to suffer from atypical hemolytic uremic syndrome (aHUS); and (ii) generating, using machine learning (ML) algorithm selected from elastic net, classification tree, and random forest, a positive predictive value (PPV) score from the subject's clinical features data.

Aspect 34: The CRM of any one of the foregoing or following Aspects, particularly Aspect 33, wherein the ML algorithm comprises elastic net and the and the clinical features data includes (1) hospitalization, (2) progression of renal disease, (3) cardiovascular/pulmonary complications, and/or (4) absence of bacterial infections.

Aspect 35: A method for identifying a subject that is suffering from or is likely to suffer from atypical hemolytic uremic syndrome (aHUS), the method comprising:
receiving data comprising a subject's clinical features selected from renal features, pulmonary features, cardiovascular features, hematology features or a combination thereof; and
generating, using machine learning (ML) algorithm selected from elastic net, classification tree, and random forest, a positive predictive value (PPV) score from the subject's clinical features data, wherein if the PPV score is above a threshold value, the subject is identified as suffering from or being likely to suffer from aHUS.

Aspect 36: The method of any one of the foregoing or following Aspects, particularly Aspect 35, wherein the ML algorithm comprises elastic net and the clinical features data includes (1) hospitalization, (2) progression of renal disease, (3) cardiovascular/pulmonary complications, and/or (4) absence of bacterial infections.

Aspect 37: The method of any one of the foregoing or following Aspects, particularly Aspect 1, wherein the diagnostic records comprise physician notes.

Aspect 38: The method of any one of the foregoing or following Aspects, particularly Aspect 37, wherein the physician notes comprise at least one of a mention of aHUS or confirmed full treatment of aHUS, wherein the mention of aHUS does not include at least one of a negative sentiment or mention of thrombocytopenia purpura (TTP).

Aspect 39: The method of any one of the foregoing or following Aspects, particularly Aspect 1, wherein the features of the machine learning model comprise one or more of multiple concordant HUS diagnoses, end-stage renal disease (ESRD), dialysis, pleurisy, lung disease, alveolar pneumopathy, pericarditis, beta blockers, cardiomegaly, thrombophlebitis, hyperphosphatemia, immune deficiency, multiple sclerosis, a chronic kidney disease, non-autoimmune hemolytic anemia, history of blood diseases, epilepsy, STEC, bacterial infections, antibiotic use, or obesity.

Aspect 40: The method of any one of the foregoing or following Aspects, particularly Aspect 39, wherein the features comprising one or more of multiple concordant HUS diagnoses, end-stage renal disease (ESRD), dialysis, pleurisy, lung disease, alveolar pneumopathy, pericarditis, beta blockers, cardiomegaly, thrombophlebitis, hyperphosphatemia, immune deficiency, or multiple sclerosis are positively associated to an aHUS diagnosis.

Aspect 41: The method of any one of the foregoing or following Aspects, particularly Aspect 39, wherein the features comprising one or more of a chronic kidney disease, non-autoimmune hemolytic anemia, history of blood diseases, epilepsy, STEC, bacterial infections, antibiotic use, or obesity are negatively associated to an aHUS diagnosis.

Aspect 42: The method of any one of the foregoing or following Aspects, particularly Aspect 1, wherein a target of the machine learning model corresponds to the aHUS related diagnosis.

What is claimed is:

1. A method for identifying a patient cohort of patients that have atypical hemolytic uremic syndrome (aHUS), comprising:
acquiring, by one or more processors, patient data for a plurality of patients, wherein the patient data comprises diagnostic records comprising an aHUS related diagnosis of each patient in the plurality of patients and claims data comprising clinical features of each patient in the plurality of patients;
determining, by the one or more processors, a subset of patients from the plurality of patients that meet inclusion criteria, wherein the inclusion criteria comprises:
claims data comprising an aHUS related code, and
diagnostic records comprising an aHUS related diagnosis;
training, by the one or more processors, a machine learning model using the subset of patients to predict whether a patient in the subset of patients has aHUS, wherein features of the machine learning model correspond to the clinical features in the claims data; and
outputting, by the one or more processors, the trained machine learning model as a predictor for identifying uncoded aHUS patients for inclusion in the patient cohort.

2. The method of claim 1, further comprising:
determining, by the trained machine learning model, whether each patient in the patient data is an uncoded aHUS patient; and
in response to a positive determination of a patient being an uncoded aHUS patient by the trained ML model, inserting, by the one or more processors, the patient into the patient cohort.

3. The method of claim 1, wherein the diagnostic records are tokenized electronic health records.

4. The method of claim 1, wherein the clinical features comprise renal features, pulmonary features, cardiovascular features, hematology features, or a combination thereof.

5. The method of claim 1, wherein the aHUS related diagnosis is at least one of hemolytic uremic syndrome (HUS) diagnosis, HUS-related hemolytic anemia diagnosis, or thrombotic microangiopathy (TMA) diagnosis.

6. The method of claim 1, wherein the aHUS related code is at least one of hemolytic uremic syndrome (HUS) codes, HUS-related hemolytic anemia codes, or thrombotic microangiopathy (TMA) codes.

7. The method of claim 1, wherein the machine learning model comprises an elastic net algorithm.

8. The method of claim 7, wherein the features of the machine learning model comprise at least one of hospitalizations, progression of renal disease, cardiovascular complications, pulmonary complications, and absence of bacterial infections.

9. The method of claim 8, wherein the features of the machine learning model comprise hospitalizations, progression of renal disease, cardiovascular complications, pulmonary complications, and absence of bacterial infections.

10. The method of claim 9, wherein the features of the machine learning model consist of hospitalizations, progression of renal disease, cardiovascular complications, pulmonary complications, and absence of bacterial infections.

11. The method of claim 1, wherein the features of the machine learning model comprise at least one of a HUS diagnosis code, a threshold number of diagnoses of HUS/TMA in the diagnostics records, occurrence of an outpatient follow-up visit for HUS/TMA, pericarditis, or immune deficiency.

12. The method of claim 11, wherein the features of the machine learning model further comprise at least one of STEC, a bacterial infection, antiepileptics medication, chronic kidney disease, or ACE inhibitors.

13. The method of claim 11, wherein the features of the machine learning model comprise a HUS diagnosis code, a threshold number of diagnoses of HUS/TMA in the diagnostics records, occurrence of an outpatient follow-up visit for HUS/TMA, pericarditis, and immune deficiency.

14. The method of claim 13, wherein the features of the machine learning model further comprise STEC, a bacterial infection, antiepileptics medication, chronic kidney disease, and ACE inhibitors.

15. The method of claim 13, wherein the features of the machine learning model consist of a HUS diagnosis code, a threshold number of diagnoses of HUS/TMA in the diagnostics records, occurrence of an outpatient follow-up visit for HUS/TMA, pericarditis, and immune deficiency.

16. The method of claim 15, wherein the features of the machine learning model consist of a HUS diagnosis code, a threshold number of diagnoses of HUS/TMA in the diagnostics records, occurrence of an outpatient follow-up visit for HUS/TMA, pericarditis, immune deficiency, STEC, a bacterial infection, antiepileptics medication, chronic kidney disease, and ACE inhibitors.

17. The method of claim 1, wherein the features of the machine learning model comprise at least one of STEC, a bacterial infection, antiepileptics medication, chronic kidney disease, or ACE inhibitors.

18. The method of claim 17, wherein the features of the machine learning model comprise STEC, a bacterial infection, antiepileptics medication, chronic kidney disease, and ACE inhibitors.

19. The method of claim 18, wherein the features of the machine learning model consist of STEC, a bacterial infection, antiepileptics medication, chronic kidney disease, or ACE inhibitors.

20. The method of claim 1, wherein the positive determination is determined by a positive predictive value that is above a threshold value.

21. A system for identifying a patient cohort of patients that have atypical hemolytic uremic syndrome (aHUS), comprising:
at least one processor; and
a memory having instructions stored thereon that, when executed by the at least one processor, cause the at least one processor to:
acquire patient data for a plurality of patients, wherein the patient data comprises diagnostic records comprising an aHUS related diagnosis of each patient in the plurality of patients and claims data comprising clinical features of each patient in the plurality of patients;
determine a subset of patients from the plurality of patients that meet inclusion criteria, wherein the inclusion criteria comprises:
claims data comprising an aHUS related code, and
diagnostic records comprising an aHUS related diagnosis;
train a machine learning model using the subset of patients to predict whether a patient in the subset of patients has aHUS, wherein the features of the machine learning model correspond to the clinical features in the claims data and the target is the aHUS related diagnosis; and
output the machine learning model as a predictor for identifying uncoded aHUS patients in claims data for inclusion in the patient cohort.

22. The system of claim 21, wherein the instructions further cause the at least one processor to:
determine whether each patient in the plurality of patients is an uncoded aHUS patient; and
in response to a positive determination of a patient being an uncoded aHUS patient, insert the patient into the patient cohort.

23. The system of claim 21, wherein the diagnostic records are electronic health records.

24. The system of claim 21, wherein the clinical features comprise renal features, pulmonary features, cardiovascular features, hematology features, or a combination thereof.

25. The system of claim 21, wherein the aHUS related diagnosis is at least one of hemolytic uremic syndrome (HUS) diagnosis, HUS-related hemolytic anemia diagnosis, and thrombotic microangiopathy (TMA) diagnosis.

26. The system of claim 21, wherein the aHUS related code is at least one of hemolytic uremic syndrome (HUS) codes, HUS-related hemolytic anemia codes, and thrombotic microangiopathy (TMA) codes.

27. The system of claim 21, wherein the machine learning model comprises an elastic net algorithm.

28. The system of claim 27, wherein the features of the machine learning model comprise at least one of hospitalizations, progression of renal disease, cardiovascular complications, pulmonary complications, and absence of bacterial infections.

29. The system of claim 21, wherein the positive determination is determined by a positive predictive value that is above a threshold value.

30. A method for identifying a patient cohort of patients that have atypical hemolytic uremic syndrome (aHUS), comprising:
predicting, by a machine learning model, whether each patient in a plurality of patients is an uncoded aHUS patient, wherein the machine learning model is trained to predict whether a patient has aHUS based on clinical features of the patient's claims data; and
in response to a positive prediction of a patient being an uncoded aHUS patient, inserting, by the one or more processors, the patient into the patient cohort.

31. A system for identifying a subject that is suffering from or is likely to suffer from atypical hemolytic uremic syndrome (aHUS), the system comprising:
a non-transitory memory; and
a hardware processor coupled with the non-transitory memory and configured to read instructions from the non-transitory memory to cause the system to perform operations comprising:
receiving data comprising a subject's clinical features selected from renal features, pulmonary features, cardiovascular features, hematology features or a combination thereof; and
generating, using machine learning (ML) algorithm selected from elastic net, classification tree, and random forest, a positive predictive value (PPV) score from the subject's clinical features data, wherein if the PPV score is above a threshold value, the subject is identified as suffering from or being likely to suffer from aHUS.

32. The system of claim 31, wherein the ML algorithm comprises elastic net and the clinical features data includes (1) hospitalization, (2) progression of renal disease, (3) cardiovascular/pulmonary complications, and/or (4) absence of bacterial infections.

33. A non-transitory computer-readable medium (CRM) having stored thereon computer-readable instructions executable to cause a computer system to perform operations comprising: (i) receiving data comprising a subject's clinical features selected from renal features, pulmonary features, cardiovascular features, hematology features or a combination thereof, wherein the subject is suffering from or is likely to suffer from atypical hemolytic uremic syndrome (aHUS); and (ii) generating, using machine learning (ML) algorithm selected from elastic net, classification tree, and random forest, a positive predictive value (PPV) score from the subject's clinical features data.

34. The non-transitory computer media of claim 33, wherein the ML algorithm comprises elastic net and the and the clinical features data includes (1) hospitalization, (2) progression of renal disease, (3) cardiovascular/pulmonary complications, and/or (4) absence of bacterial infections.

35. A method for identifying a subject that is suffering from or is likely to suffer from atypical hemolytic uremic syndrome (aHUS), the method comprising:
receiving data comprising a subject's clinical features selected from renal features, pulmonary features, cardiovascular features, hematology features or a combination thereof; and
generating, using machine learning (ML) algorithm selected from elastic net, classification tree, and random forest, a positive predictive value (PPV) score from the subject's clinical features data, wherein if the PPV score is above a threshold value, the subject is identified as suffering from or being likely to suffer from aHUS.

36. The method of claim 35, wherein the ML algorithm comprises elastic net and the clinical features data includes (1) hospitalization, (2) progression of renal disease, (3) cardiovascular/pulmonary complications, and/or (4) absence of bacterial infections.

37. The method of claim 1, wherein the diagnostic records comprise physician notes.

38. The method of claim 37, wherein the physician notes comprise at least one of a mention of aHUS or confirmed full treatment of aHUS, wherein the mention of aHUS does not include at least one of a negative sentiment or mention of thrombocytopenia purpura (TTP).

39. The method of claim 1, wherein the features of the machine learning model comprise one or more of multiple concordant HUS diagnoses, end-stage renal disease (ESRD), dialysis, pleurisy, lung disease, alveolar pneumopathy, pericarditis, beta blockers, cardiomegaly, thrombophlebitis, hyperphosphatemia, immune deficiency, multiple sclerosis, a chronic kidney disease, non-autoimmune hemolytic anemia, history of blood diseases, epilepsy, STEC, bacterial infections, antibiotic use, or obesity.

40. The method of claim 39, wherein the features comprising one or more of multiple concordant HUS diagnoses, end-stage renal disease (ESRD), dialysis, pleurisy, lung disease, alveolar pneumopathy, pericarditis, beta blockers, cardiomegaly, thrombophlebitis, hyperphosphatemia, immune deficiency, or multiple sclerosis are positively associated to an aHUS diagnosis.

41. The method of claim 39, wherein the features comprising one or more of a chronic kidney disease, non-autoimmune hemolytic anemia, history of blood diseases, epilepsy, STEC, bacterial infections, antibiotic use, or obesity are negatively associated to an aHUS diagnosis.

42. The method of claim 1, wherein a target of the machine learning model corresponds to the aHUS related diagnosis.
